(19) United States
(12) Patent Application Publication
(10) Pub. No.: US 2026/0183473 A1
(43) Pub. Date: Jul. 2, 2026

(54) MEDICAL DISPENSING DEVICE

(71) Applicant: Roche Diabetes Care, Inc., Indianapolis, IN (US)

(72) Inventor: Harald von Campenhausen, Ladenburg (DE)

(21) Appl. No.: 19/127,355

(22) PCT Filed: Nov. 3, 2023
(86) PCT No.: PCT/EP2023/080641

§ 371 Date: May 5, 2025

(30) Foreign Application Priority Data
Nov. 5, 2022 (EP) 22205675.6

(51) Int. Cl. A61M 5/14; A61M 5/142; A61M 5/145; A61M 5/158; A61M 5/172

(52) U.S. Cl. CPC A61M 5/1408; A61M 5/14244; A61M 5/1452; A61M 5/172; A61M 2005/1585; A61M 2209/01; A61M 2209/088

(57) ABSTRACT

The present invention relates to a medical dispensing device comprising a first cylindrical reservoir, a second cylindrical reservoir, a first plunger movably arranged within the first cylindrical reservoir, a second plunger movably arranged within the second cylindrical reservoir, and a drive that is configured for moving the first plunger within the first cylindrical reservoir and that is further configured for moving the second plunger within the second cylindrical reservoir. For an improved dosing accuracy, the drive is configured to selectively move either the first plunger or the second plunger, and in that the drive is further configured to stop moving the first plunger within the first cylindrical reservoir when the first plunger reaches the ending point within the first cylindrical reservoir, and to start moving the second plunger within the second cylindrical reservoir.

The present invention relates to a medical dispensing device comprising a first cylindrical reservoir that is configured for containing a liquid medicament, a second cylindrical reservoir that is configured for containing a liquid medicament, a first plunger that is movably arranged within the first cylindrical reservoir between a first plunger starting point and a first plunger ending point, wherein with the first plunger at the first plunger starting point the volume for the liquid medicament within the first cylindrical reservoir delimited by the first plunger is maximum and with the first plunger at the first plunger ending point the volume for the liquid medicament within the first cylindrical reservoir delimited by the first plunger is minimum, a second plunger that is movably arranged within the second cylindrical reservoir between a second plunger starting point and a second plunger ending point, wherein with the second plunger at the second plunger starting point the volume for the liquid medicament within the second cylindrical reservoir delimited by the second plunger is maximum and with the second plunger at the second plunger ending point the volume for the liquid medicament within the second cylindrical reservoir delimited by the second plunger is minimum, and a drive that is configured for moving the first plunger within the first cylindrical reservoir and that is further configured for moving the second plunger within the second cylindrical reservoir.

A hitherto known medical dispensing device is used for administering a liquid medicament to a user. The liquid medicament is intended to improve the patient's health condition. During use, liquid medicament is simultaneously dispensed out of the first cylindrical reservoir and out of the second cylindrical reservoir. Like any other dispensing process, dispensing liquid medicament out of the first cylindrical reservoir is prone to an inaccurate metering inherent to the dosing mechanism of the first cylindrical reservoir and dispensing liquid medicament out of the second cylindrical reservoir is prone to an inaccurate metering inherent to the dosing mechanism of the second cylindrical reservoir. By simultaneously dispensing liquid medicament out of the first cylindrical reservoir and out of the second cylindrical reservoir the system inherent inaccurate metering tied to the first cylindrical reservoir and the system inherent inaccurate metering tied to the second cylindrical reservoir add-up. The added-up system inherent inaccurate metering may result in an underdosing or overdosing of liquid medicament, which, dependent of the type of liquid medicament, can cause a severe health risk to the user of the medical dispensing device. Also known are medical dispensing devices which comprise only one cylindrical reservoir and which are associated with the disadvantage that the overall reservoir volume is either too small or the device becomes too bulky in which case users find it inconvenient to carry such bulky devices in a pocket or on their skin. Moreover, large volume reservoirs are also associated with inherent inaccurate metering.

Accordingly, it is an object of the present invention to provide a medical dispensing device which avoid at least one disadvantage of hitherto known medical dispensing devices. In particular, it is an object of the present invention to provide a medical dispensing device having an improved dosing accuracy and at the same time offer a pump with a large reservoir volume.

At least one of the above objects is solved by the provision of the subject matter disclosed in the claims and throughout the specification.

According to a first aspect, a medical dispensing device comprising a first cylindrical reservoir that is configured for containing a liquid medicament, a second cylindrical reservoir that is configured for containing a liquid medicament, a first plunger that is movably arranged within the first cylindrical reservoir between a first plunger starting point and a first plunger ending point, wherein with the first plunger at the first plunger starting point the volume for the liquid medicament within the first cylindrical reservoir delimited by the first plunger is maximum and with the first plunger at the first plunger ending point the volume for the liquid medicament within the first cylindrical reservoir delimited by the first plunger is minimum, a second plunger that is movably arranged within the second cylindrical reservoir between a second plunger starting point and a second plunger ending point, wherein with the second plunger at the second plunger starting point the volume for the liquid medicament within the second cylindrical reservoir delimited by the second plunger is maximum and with the second plunger at the second plunger ending point the volume for the liquid medicament within the second cylindrical reservoir delimited by the second plunger is minimum, and a drive that is configured for moving the first plunger within the first cylindrical reservoir and that is further configured for moving the second plunger within the second cylindrical reservoir, wherein the drive is configured to selectively move either the first plunger or the second plunger, and in that the drive is further configured to stop moving the first plunger within the first cylindrical reservoir when the first plunger reaches the first plunger ending point within the first cylindrical reservoir, and to start moving the second plunger within the second cylindrical reservoir.

The first cylindrical reservoir and the second cylindrical reservoir are configured for containing a liquid medicament. The first cylindrical reservoir and the second cylindrical reservoir provide the liquid medicament which, during use of the medical dispensing device, can be administered to a user of the medical dispensing device. The first cylindrical reservoir and the second cylindrical reservoir, both, comprise a circular cross-sectional area.

The first plunger is movably arranged within the first cylindrical reservoir between a first plunger starting point and a first plunger ending point. When moving the first plunger from the first plunger starting point in a direction towards the first plunger ending point the volume delimited by the inner walls of the first cylindrical reservoir and the first plunger is decreased, such that liquid medicament contained in the shrinking volume is dispensed out of the first cylindrical reservoir. In a state, in which the first plunger is located at the first plunger starting point the volume for the liquid medicament within the first cylindrical reservoir is maximum, while in a state, in which the first plunger is located at the first plunger ending point the volume for the liquid medicament within the first cylindrical reservoir is minimum. For instance, the minimum volume can be zero or close to zero, such that the first plunger abuts against the inner, circular wall defining an end of the first cylindrical reservoir.

The second plunger is movably arranged within the second cylindrical reservoir between a second plunger starting point and a second plunger ending point. When moving the second plunger from the second plunger starting point in a direction towards the second plunger ending point the volume delimited by the inner walls of the second cylindrical reservoir and the second plunger is decreased, such that liquid medicament contained in the shrinking volume is dispensed out of the second cylindrical reservoir. In a state, in which the second plunger is located at the second plunger starting point the volume for the liquid medicament within the second cylindrical reservoir is maximum, while in a state, in which the second plunger is located at the second plunger ending point the volume for the liquid medicament within the second cylindrical reservoir is minimum. For instance, the minimum volume can be zero or close to zero, such that the second plunger abuts against the inner, circular wall defining an end of the second cylindrical reservoir.

According to an embodiment, the maximum volume of each of the first cylindrical reservoir and the second cylindrical reservoir can be between 1 to 100 ml, preferable between 1 to 50 ml, preferably between 1 to 40 ml, preferable between 1 to 30 ml, preferable between 1 to 20 ml, preferable between 1 to 10 ml, preferable between 1 to 5 ml, and preferable between 1 to 3 ml.

The dosing accuracy are inherent properties of the first cylindrical reservoir and the second cylindrical reservoir. The accuracy is determined by the underlying constructions, mechanisms and electronics of the first and the second reservoir, respectively, as well as any further component of the medical dispensing device system needed for delivering a dosage of liquid medicament by operating the first and the second reservoir, such as the plunger and the drive. By distributing the total liquid volume capacity of the medical dispensing device to two separate reservoirs an increase in dosing accuracy is achieved while at the same time avoiding a bulky form of the medical dispensing device that is associated with single-reservoir medical dispensing devices.

The drive of the medical dispensing device is configured for moving the first plunger within the first cylindrical reservoir, for instance in a direction from the first plunger starting point towards the first plunger ending point. The drive is further configured for moving the second plunger within the second cylindrical reservoir, for example in a direction from the second plunger starting point towards the second plunger ending point. In an embodiment, the drive is configured to move the first plunger and the second plunger continuously or stepwise.

The drive can comprise a motor that is configured to move the first plunger within the first cylindrical reservoir in a direction from the first plunger starting point towards the first plunger ending point and to move the second plunger within the second cylindrical reservoir in a direction from the second plunger starting point towards the second plunger ending point. The drive can comprise a single motor that is configured for coupling at a time either to the first plunger rod or to the second plunger rod. Providing a single motor for either driving the first plunger by the first plunger rod or the second plunger by the second plunger rod, is in particular beneficial for assuring that only one of the first plunger and the second plunger is moved in a direction from the respective first plunger starting point or second plunger starting point to the first plunger ending point or second plunger ending point. In addition a single motor-type construction is less costly, more energy efficient and lighter in weight compared to 2-motor constructions.

Alternatively, the drive can comprise a first motor and a second motor, wherein the first motor is configured to move the first plunger within the first cylindrical reservoir in a direction from the first plunger starting point towards the first plunger ending point and the second motor is configured to move the second plunger within the second cylindrical reservoir in a direction from the second plunger starting point towards the second plunger ending point. While the first plunger is advanced from the first plunger starting point towards the first plunger ending point within the first cylindrical reservoir by moving the first plunger with the first motor, the second motor is in an idle state. In an idle state, the second motor provides a holding force that holds the second plunger in place, such that an unintentional movement of the second plunger within the second cylindrical reservoir is prevented. The same principle applies, in case the second motor drives the second plunger to move within the second cylindrical reservoir in a direction from the second plunger starting point towards the second plunger ending point, while the first motor is in an idle state. The first motor in an idle state helps to prevent an unintentional movement of the first plunger within the first cylindrical reservoir, while the second motor drives the second plunger within the second cylindrical reservoir in a direction from the second plunger starting point towards the second plunger ending point. For instance, each of the one or more motors can be an electric motor, in particular a stepper motor.

At any time, either the first plunger or the second plunger is moved by the drive, wherein, in the beginning, liquid medicament is dispensed out of the first cylindrical reservoir by advancing the first plunger within the first cylindrical reservoir by the drive from the first plunger starting point in the direction towards the first plunger ending point. While the first plunger is moved within the first cylindrical reservoir in a direction from the first plunger starting point to the first plunger ending point, the second plunger is not moved within the second cylindrical reservoir. Once the first plunger reaches the first plunger ending point within the first cylindrical reservoir, the movement of the first plunger stops. A further or remaining demand of liquid medicament will be dispensed by operating the second cylindrical reservoir. I.e. after the first plunger has reached the first plunger ending point within the first cylindrical reservoir, the drive moves the second plunger within the second cylindrical reservoir in a direction from the second plunger starting point to a second plunger ending point. Accordingly, the first reservoir and the second reservoir are subsequently depleted in series. The system's inherent inaccurate metering tied to the first cylindrical reservoir and the system's inherent inaccurate metering tied to the second cylindrical reservoir do not add-up.

Subsequently depleting in series the first cylindrical reservoir and the second cylindrical reservoir is also advantageous, as it can also help to maintain the overall quality of the liquid medicament as dispensed by the medical dispensing device. During use of the medical dispensing device, bodily-fluids or other impurities may reach the first cylindrical reservoir. However, as first the cylindrical reservoir is used for dispensing liquid medicament, bodily fluids or other impurities will be restricted to the content of the first cylindrical reservoir as the second cylindrical reservoir is not yet used. Accordingly, the quality of the liquid medicament within the second cylindrical reservoir is preserved until the first cylindrical reservoir is depleted and liquid medicament is dispensed out of the second cylindrical reservoir.

The movement of the first plunger as well as the movement of the second plunger can be continuous or interrupted, such that liquid medicament is dispensed out of the respective first cylindrical reservoir and second cylindrical reservoir in one more portions of liquid medicament.

The medical dispensing device can be a body-wearable medical device, such as for example a patch pump which can be releasably attached to a patient's skin at an insertion site by an adhesive or a body-mounting device. The medical dispensing device can comprise a transcutaneous element, for example a steel cannula or a soft cannula, that can be at least partially inserted into the patient's body.

For example, the liquid medicament can be any liquid drug suited for transcutaneous administration including insulin or glucagon.

According to an embodiment, the drive is configured to block movement of the second plunger until the first plunger reaches the first plunger ending point in the first cylindrical reservoir. Dispensing of liquid medicament begins by moving the first plunger within the first cylindrical reservoir by the drive in a direction from the first plunger starting point towards the first plunger ending point. To assure that the first cylindrical reservoir is depleted first, movement of the second plunger within the second cylindrical reservoir is blocked until the first plunger reaches the first plunger ending point in the first cylindrical reservoir. The drive can be configured to release the blocking of the movement of the second plunger once the first plunger reaches the first plunger ending point in the first cylindrical reservoir.

The drive can be configured to block movement of the second plunger within the second cylindrical reservoir while the first plunger is moving within the first cylindrical reservoir and/or the drive can be configured to block movement of the first plunger within the first cylindrical reservoir while the second plunger is moving within the second cylindrical reservoir. Blocking the movement of the second plunger while the first plunger is moving within the first cylindrical reservoir helps to increase the safeness of the medical dispensing device. When being blocked, unintentional and uncoordinated flow of liquid medicament out of the second cylindrical reservoir is prevented. Furthermore, blocking the movement of the second plunger also prevents that the second plunger is unintentionally moving in a direction from the second plunger ending point towards the second starting point. Otherwise, when being unintentionally moved in a direction from the second plunger ending point towards the second plunger starting point a negative pressure can be generated within the second cylindrical reservoir. The negative pressure may cause an unintentionally flow of liquid medicament or bodily fluids into the second cylindrical reservoir. Thus, blocking the movement of the second plunger within the second cylindrical reservoir, while the first plunger is moved by the drive within the first cylindrical reservoir, increase the accuracy of the dispensing of liquid medicament as liquid medicament dispensed out of the first reservoir is not unintentionally drawn into the second cylindrical reservoir. Likewise, blocking the movement of the first plunger within the first cylindrical reservoir while the second plunger is moving also helps to increase the safeness of the medical dispensing device as due to the blocking of the movement of the first plunger no liquid medicament or bodily fluids is unintentionally drawn into the first cylindrical reservoir.

The drive can comprise a mechanical blocking element. For example, a non-limited list of mechanical blocking elements comprises a catch, a pawl, and a ratchet. In addition or alternatively, the drive can comprise an electrical or electromagnetic blocking element. The blocking element can be configured to be brought in a blocking position and configured to be removed from the blocking position. However, in an embodiment the blocking element is configured to irreversibly block a movement of the first plunger within the first cylindrical reservoir when the first plunger reaches the first plunger ending point and/or the blocking element is configured to irreversibly block a movement of the second plunger within the second cylindrical reservoir when the second plunger reaches the second plunger ending point within the second cylindrical reservoir. Thereby, neither the first cylindrical reservoir nor the second cylindrical reservoir can be reused after being depleted. “Irreversible” in the meaning of the present invention means that the blocking element cannot be removed from its respective blocking position without manipulating the medical dispensing device or parts thereof to operate in contrast to its purpose. In particular, the blocking element cannot be removed from its respective blocking position without damaging or destroying the medical dispensing device or parts thereof.

According to an embodiment, the medical dispensing device comprises a first plunger rod configured to engage the first plunger, and a second plunger rod that is configured to engage the second plunger, wherein the drive is configured to selectively actuate, either the first plunger rod or the second plunger rod. During use of the medical dispensing device, the first plunger rod transfers a movement that is induced by the drive to the first plunger, such that the first plunger moves within in the first cylindrical reservoir in a direction from the first plunger starting point towards the first plunger ending point. Likewise, the second plunger rod transfers a movement induced by the drive to the second plunger, such that the second plunger moves within the second cylindrical reservoir in a direction from the second plunger starting point towards the second plunger ending point. The drive can be continuously or selectively engaged to the first plunger rod and the second plunger rod. Providing a first plunger rod and a second plunger rod allows to the drive to be stationary while the respective first plunger and second plunger are moving relative to drive by the help of the first plunger rod and the second plunger rod.

The drive can comprise a hinge and a coupling element, wherein the coupling element is configured to couple the drive alternatively to the first plunger or to the second plunger, and wherein the hinge is configured to selectively couple by a rotational movement either the coupling element to the first plunger and to decouple the coupling element from the second plunger, or to decouple the coupling element from the first plunger and to couple the coupling element to the second plunger. The coupling element transmits a movement that is induced by the drive either to the first plunger or to the second plunger. A non-exhaustive list of examples of a coupling element comprise a gearwheel, a clutch, or a magnetic coupling. There can be more than one coupling element. For instance, there can be a first coupling element that is configured to couple the first plunger to the drive and to decoupled the first plunger from the drive, respectively, and a second coupling element that is configured to couple the second plunger to the drive and to decouple the second plunger from the drive, respectively. Due to the hinge the coupling element is either coupled to the first plunger or to the second plunger. The coupling element is moved by the hinge so to engage or disengage the respective first or second plunger. The coupling element can be directly coupled to the first plunger or the second plunger, respectively or the coupling element can be indirectly coupled to the first plunger or the second plunger, respectively, via a third or further elements. For instance, the coupling element can comprise a gear wheel for selectively engaging a first plunger rod and a second plunger rod. Alternatively, the coupling can comprise a first gear wheel for engaging a first plunger rod and a second gear wheel for engaging the second plunger rod. The first plunger rod and the second plunger rod can be configured to be coupled to the first plunger or second plunge, respectively, as described above.

The drive can comprise a sliding mechanism and a coupling element, wherein the coupling element is configured to couple the drive alternatively to the first plunger or the second plunger, and wherein the sliding mechanism is configured, by a sliding movement, either, at a time, to couple the coupling element to the first plunger and to decouple the coupling element from the second plunger, or to decouple the coupling element from the first plunger and to couple the coupling element to the second plunger. Again the coupling element transmits a movement induced from the drive to the respective first plunger or second plunger currently coupled via the coupling element to the drive. By a shifting movement of the shifting element the coupling element is coupled to the first plunger and decoupled from the second plunger. Likewise, by a shifting movement of the shifting element in an opposite direction, the coupling element is decoupled from the first plunger and coupled to the second plunger. Thus, the sliding element allows at least a first state in which the first plunger is coupled to the drive via the coupling element while the second plunger is decoupled from the drive, and a second state in which the first plunger is decoupled from the drive and the second plunger is coupled to the drive via the coupling element. There can be a third state in which neither the first plunger is coupled to the drive nor the second plunger is coupled to the drive. The coupling element can be directly coupled to the first plunger or the second plunger, respectively or the coupling element can be indirectly coupled to the first plunger or the second plunger, respectively, via a third or further elements. For instance, the coupling element can be coupled or decoupled from a first plunger rod, and can be coupled or decoupled from a second plunger rod, wherein the first plunger rod is configured to transmit a movement from the drive to the first plunger so to move the first plunger within the first cylindrical reservoir and the second plunger rod is configured to transmit a movement from the drive to the second plunger so to move the second plunger within the second cylindrical reservoir. The drive can comprise the sliding mechanism additionally or alternatively to comprising a hinge. When comprising the sliding mechanism and the hinge, the same coupling element can be used to couple the drive, at any time, alternatively to the first plunger or the second plunger by a combined sliding and rotational movement.

The first cylindrical reservoir and the second cylindrical reservoir of the medical dispensing device can be oriented antiparallel to each other, wherein the drive comprises a driving element that is configured for moving the first plunger within in the first cylindrical reservoir in a direction from the first plunger starting point towards the first plunger ending point by a rotational movement in a first direction and that is configured for moving the second plunger within the second cylindrical reservoir in a direction from the second plunger starting point towards the second plunger ending point by a rotational movement in a second direction opposite to the first direction. Being antiparallel to each other, the direction of moving the first plunger within the first cylindrical reservoir from the first plunger starting point towards the first plunger ending point is opposite to the direction of moving the second plunger within the second cylindrical reservoir from the second plunger starting point towards the second plunger ending point. Thereby it is possible to selectively move the first plunger and the second plunger by selecting the direction of movement of the driving element. For example, the driving element can be configured for moving the first plunger within the first cylindrical reservoir by a rotation movement in a first direction, and further configured for moving the second plunger within the second cylindrical reservoir by a rotational movement in a second direction, opposite to the first rotational movement.

The present invention further relates to a medical dispensing system comprising a medical dispensing device as described herein, and at least one selected from the group of a remote control configured to communicate with the medical dispensing device and/or control the medical dispensing device, an infusion set device configured to couple to the medical dispensing device and wherein the infusion set device is configured to convey liquid medicament received from the medical dispensing device via an invasive infusion cannula into a patient's body, a body mount configured to attach the medical dispensing device to the skin of a user, an insertion device configured to insert an invasive cannula at least in part into the patient's body, and a rechargeable battery. The remote control configured to communicate with the medical dispensing device transmits data to and/or receives data from the medical dispensing device. For instance, the transmittal of data is achieved by using a wireless data transfer protocol. A remote control that in addition or alternatively is configured to control the medical dispensing device transmits data triggering a control command and/or control commands to the medical dispensing device. During use, an infusion set device configured to couple to the medical dispensing device receives liquid medicament from the medical dispensing device and conveys the liquid medicament via an invasive infusion cannula into a patient's body. A body mount configured to attach the medical dispensing device to the skin of a user helps to carry the medical dispensing device close to the user's body. Furthermore, the body mount can be configured to removably attach the medical dispensing device to a user's body. For instance, the body mount can be releasably attached to the user's body by an adhesive, and the medical dispensing device can be releasably mounted to the body mount by corresponding engaging elements, like for example a latch and a corresponding catch. An insertion device configured to insert an invasive infusion cannula at least in part into the patient's body provides a mechanism for automatically or semi-automatically introduce an invasive infusion cannula at least in part into the patient's body. The medical dispensing device system can comprise a rechargeable battery which provides energy for operating the medical dispensing device system or parts thereof, as for example the medical dispensing device.

The medical dispensing device can be mounted on the infusion set device. For example, the infusion set device can form a body-mount configured to attach the medical dispensing device to the skin of a user and at the same time is configured to convey liquid medicament dispensed from the medical dispensing device via an invasive infusion cannula into a patient's body.

Further advantages, features and technical effects of the present invention are apparent from the following description of embodiments and the accompanying figures.

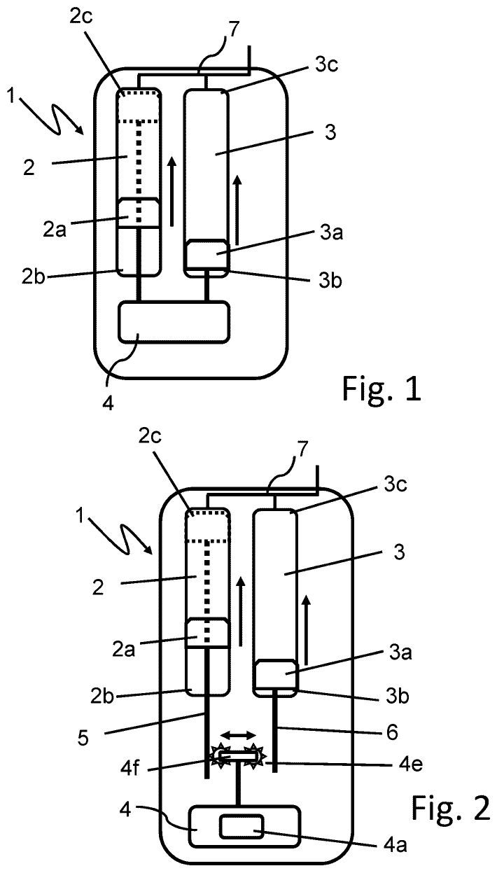

FIG. 1 shows a schematic view of a medical dispensing device according to an embodiment of the invention;

FIG. 2 shows a schematic view of a medical dispensing device according to another embodiment of the invention;

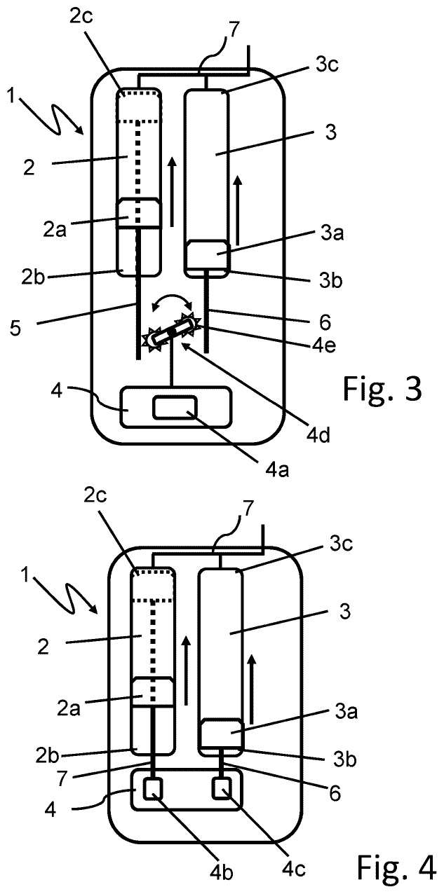

FIG. 3 shows a schematic view of a medical dispensing device according to another embodiment of the invention; and

FIG. 4 shows a schematic view of a medical dispensing device according to another embodiment of the invention.

FIG. 1 shows a medical dispensing device 1 comprising a first cylindrical reservoir 2 and a second cylindrical reservoir 3. The first cylindrical reservoir 2 and the second cylindrical reservoir 3 are both configured for containing liquid medicament. Here for instance, the medical dispensing device 1 is an insulin patch pump, and the liquid medicament is insulin.

A first plunger 2a is movably arranged within the first cylindrical reservoir 2. The first plunger 2a can be moved from a first plunger starting point 2b towards a first plunger ending point 2c. In case the first plunger 2a is at the first plunger starting point 2b, the volume for the liquid medicament within the first cylindrical reservoir 2 is maximum, wherein in case the first plunger 2a is at the first plunger ending point 2c the volume for the liquid medicament within the first cylindrical reservoir 2 is minimum. When advancing the first plunger 2a from the first plunger starting point 2b towards the first plunger ending point 2c, the volume for the liquid medicament is decreased, such that liquid medicament contained in the first cylindrical reservoir 2 is dispensed through a fluid line 7. The end of the fluid line 7 can be connected to a cannula or can form a cannula.

Likewise, a second plunger 3a is movably arranged within the second cylindrical reservoir 3. The second plunger 3a can be moved from a second plunger starting point 3b towards a second plunger ending point 3c. In case the second plunger 3a is at the second plunger starting point 3b, the volume for the liquid medicament within the second cylindrical reservoir 3 is maximum, wherein in case the second plunger 3a is at the second plunger ending point 3c the volume for the liquid medicament within the second cylindrical reservoir 3 is minimum. When advancing the second plunger 3a from the second plunger starting point 3b towards the second plunger ending point 3c, the volume for the liquid medicament is decreased, such that liquid medicament contained in the second cylindrical reservoir 3 is dispensed through the same fluid line 7 as liquid medicament that is dispensed from the first cylindrical reservoir 2.

The movability of the first plunger 2a and the second plunger 3a is indicated by broken-lines showing the first plunger 2a and the second plunger 3a in different positions within the respective first cylindrical reservoir 2 and second cylindrical reservoir 3.

The medical dispensing device 1 further comprises a drive 4. Drive 4 is configured for moving the first plunger 2a within the first cylindrical reservoir 2 and for moving the second plunger 3a within the second cylindrical reservoir 3. For allowing the first cylindrical reservoir 2 and the second cylindrical reservoir 3 to be subsequently depleted, the drive 4 is further configured to selectively move either the first plunger 2a or the second plunger 3a. Accordingly, during use only one of the first plunger 2a or the second plunger 3a is moving, while the other one of the first plunger 2a and second plunger 3a is not moving, but maintains its position within the respective cylindrical reservoir 2, 3.

Firstly, liquid medicament will be dispensed out of the first cylindrical reservoir 2 by moving the first plunger 2a within the first cylindrical reservoir 2 in a direction from the first plunger starting point 2b towards the first plunger ending point 2c. While moving the first plunger 2a, the second plunger 3 maintains its position within the second cylindrical reservoir 3, so to prevent an unintentional flow of liquid medicament out of or into the second cylindrical reservoir 3. Upon the first plunger 2a reaching the first plunger ending point 2c within the first cylindrical reservoir 2, the drive 4 stops moving the first plunger 2a, but starts dispensing of liquid medicament out of the second cylindrical reservoir 3 by moving the second plunger 3a within the second cylindrical reservoir 3 in a direction from the second plunger starting point 3b towards the second plunger ending point 3c. The position of the first plunger 2a within the first cylindrical reservoir is maintained. The first plunger 2a and the second plunger 3a can be moved continuously or incrementally, depending on the amount of liquid medicament to be dispensed by the medical dispensing device.

FIG. 2 shows a schematic view of medical dispensing device 1 according to another embodiment. The medical dispensing device 1 comprises a first cylindrical reservoir 2 and a second cylindrical reservoir 3. Again, a first plunger 2a is movably arranged within the first cylindrical reservoir 2. When being at a first plunger starting point 2b the volume for liquid medicament as delimited by the first plunger 2a within the first cylindrical reservoir 2 is maximum, while when being at a first plunger ending point 2c the volume for liquid medicament as delimited by the first plunger 2a within the first cylindrical reservoir 2 is minimum.

Likewise, a second plunger 3a is movably arranged within the second cylindrical reservoir 3. Being at a second plunger starting point 2b the volume for liquid medicament as delimited by the second plunger 3a within the second cylindrical reservoir 3 is maximum, while when being at a second plunger ending point 3c the volume for liquid medicament as delimited by the second plunger 3a within the second cylindrical reservoir 3 is minimum.

The medical dispensing device further comprises a drive 4 that is configured for advancing the first plunger 2a within the first cylindrical reservoir 2 in a direction from the first plunger starting point 2b towards the first plunger ending point 2c and for advancing the second plunger 3a within the second cylindrical reservoir 3 in a direction from the second plunger starting point 3b towards the second plunger ending point 3c.

Drive 4 comprises a motor 4a. Motor 4a is configured to move the first plunger 2a within the first cylindrical reservoir 2 and to move the second plunger 3a within the second cylindrical reservoir 3. More specific, the medical dispensing device 1 comprises a first plunger rod 5 and a second plunger rod 6. The first plunger rod 5 is operatively coupled to the first plunger 2a, so that by actuating the first plunger rod 5 the first plunger 2a is moved within the first cylindrical reservoir 2. Likewise, the second plunger rod 6 is operatively coupled to the second plunger 3a. Accordingly, by actuating the second plunger rod 6 the second plunger 3a is moved within the second cylindrical reservoir 3.

For actuating the first plunger rod 5 or the second plunger rod 6, i.e. only one of the first plunger rod 5 and the second plunger rod 6 at a time is actuated by the motor 4a, drive 4 further comprises a sliding mechanism 4f and a coupling element 4e. The coupling element 4f of this embodiment comprises two gears. One of the two gears is configured to engage the first plunger rod 5, and the other one of the two gears is configured to engage the second plunger rod 6. In order to engage either first plunger rod 5 or the second plunger rod 6, the coupling element 4e is displaceable by the sliding mechanism 4f. At a time, by the sliding mechanism either the first plunger rod 5 is coupled by the coupling element 4e to the motor 4a of the drive 4, or the second plunger rod 6 is coupled by the coupling element 4e to the motor 4a of the drive 4. Accordingly, the sliding mechanism 4f is configured to selectively couple by a sliding movement either the coupling element 4e to the first plunger 2a or the coupling element 4e to the second plunger 3a. In a state, in which the first plunger 2a is coupled to the motor 4a, the coupling element 4e is in engagement with the first plunger rod 5 while the coupling element 4e is in disengagement with the second plunger rod 6. Likewise, in a state in which the second plunger 2a is coupled to the motor 4a of the drive 4, the coupling element 4e and the first plunger rod 5 are disengaged, while the coupling element 4e engages the second plunger rod 6.

FIG. 3 shows another embodiment of a medical dispensing device 1. This embodiment of the medical dispensing device 1 differs from the embodiment of a medical dispensing device 1 as shown in FIG. 2 in the configuration of drive 4. Drive 4 also comprises a motor 4a that is configured for actuating the first plunger rod 5 so to move the first plunger 2a within the first cylindrical reservoir 2 in a direction from the first plunger starting point 2b to the first plunger ending point 2c, and that is configured actuating the second plunger rod 6 so to move the second plunger 3a within the second cylindrical reservoir 3 in a direction from the second plunger starting point 3b to the second plunger ending point 3c. However, instead of comprising a sliding mechanism 4f, the drive 4 of the embodiment of a medical dispensing device as shown in FIG. 3 comprises a hinge 4d.

Hinge 4d is configured to selectively couple by a turning movement either the coupling element 4e to the first plunger 2a, here via the first plunger rod 5, or to the second plunger 3a, here via the second plunger rod 6. In a state, in which the coupling element 4e is coupled to the first plunger rod 5, the coupling element 4e is decoupled from the second plunger rod 6, such that the first plunger 2a is coupled to the motor 4a while the second plunger 3a is decoupled from the motor 4a. Accordingly, in a state, in which the coupling element 4e is coupled to the second plunger rod 6, the coupling element 4e is decoupled from the first plunger rod 5, such that the first plunger 2a is decoupled from the motor 4a while the second plunger 3a is coupled to the motor 4a. Thereby, the first plunger 2a can be moved within the first cylindrical reservoir 2 while the second plunger 3a is not moved in the second cylindrical reservoir, and vice versa.

FIG. 4 shows yet another embodiment of a medical dispensing device 1. The medical dispensing device 1 as shown in FIG. 4 differs from the embodiment of a medical dispensing device according to FIG. 3 by not comprising a hinge 4d and a coupling element 4e associated to the hinge 4e, but by comprising a drive 4 that comprises a first motor 4b and a second motor 4c. The first motor 4b is configured to actuate the first plunger rod 5 so to move the first plunger 2a within the first cylindrical reservoir 2 in a direction from the first plunger starting point 2b to the first plunger ending point 2c. Likewise, the second motor 4c is configured to actuate the second plunger rod 6 so to move the second plunger 3a within the second cylindrical reservoir 3 in a direction from the second plunger starting point 3b to the second plunger ending point 3c. The first motor 4b is further configured to block motion of the first plunger 2a by blocking a movement of the first plunger rod 5, when the first plunger 2a has reached its ending point 2c. The second motor 4c is configured to block motion of the second plunger 3a by blocking a movement of the second plunger rod 6 when the first plunger 2a has not yet reached its first plunger ending point 2c. Thereby, it is possible to prevent an unintended flow of liquid medicament out of or into the second cylindrical reservoir 3 while the first plunger 2a is moving, and to prevent an unintended flow of liquid medicament out of or into the first cylindrical reservoir 2 while the second plunger 3a is moving.

### LIST OF REFERENCE NUMBERS

1 Medical dispensing device
2 first cylindrical reservoir
2a first plunger
2b first plunger starting point
2c first plunger ending point
3 second cylindrical reservoir
3a second plunger
3b second plunger starting point
3c second plunger ending point
4 drive
4a motor
4b first motor
4c second motor
4d hinge
4e coupling element
4f sliding mechanism
5 first plunger rod
6 second plunger rod
7 fluid line

1. A medical dispensing device comprising:
a first cylindrical reservoir that is configured for containing a liquid medicament;
a second cylindrical reservoir that is configured for containing a liquid medicament;
a first plunger that is movably arranged within the first cylindrical reservoir between a first plunger starting point and a first plunger ending point, wherein with the first plunger at the first plunger starting point the volume for the liquid medicament within the first cylindrical reservoir delimited by the first plunger is maximum and with the first plunger at the first plunger ending point the volume for the liquid medicament within the first cylindrical reservoir delimited by the first plunger is minimum,
a second plunger that is movably arranged within the second cylindrical reservoir between a second plunger starting point and a second plunger ending point, wherein with the second plunger at the second plunger starting point the volume for the liquid medicament within the second cylindrical reservoir delimited by the second plunger is maximum and with the second plunger at the second plunger ending point the volume for the liquid medicament within the second cylindrical reservoir delimited by the second plunger is minimum; and
a drive that is configured for moving the first plunger within the first cylindrical reservoir and that is further configured for moving the second plunger within the second cylindrical reservoir,
wherein the drive is configured to selectively move either the first plunger or the second plunger, and wherein the drive is further configured to stop moving the first plunger within the first cylindrical reservoir when the first plunger reaches the first plunger ending point within the first cylindrical reservoir, and to start moving the second plunger within the second cylindrical reservoir.

2. The medical dispensing device according to claim 1, wherein the drive is configured to block movement of the second plunger until the first plunger reaches the ending point in the first cylindrical reservoir.

3. The medical dispensing device according to claim 1, wherein the drive is configured to block movement of the second plunger within the second cylindrical reservoir while the first plunger is moving within the first cylindrical reservoir and/or the drive is configured to block movement of the first plunger within the first cylindrical reservoir while the second plunger is moving within the second cylindrical reservoir.

4. The medical dispensing device according to claim 1, wherein the drive comprises a motor that is configured to move the first plunger within the first cylindrical reservoir in a direction from the first plunger starting point towards the first plunger ending point and to move the second plunger within the second cylindrical reservoir in a direction from the second plunger starting point towards the second plunger ending point, or in that the drive comprises a first motor and a second motor, wherein the first motor is configured to move the first plunger within the first cylindrical reservoir in a direction from the first plunger starting point towards the first plunger ending point and the second motor is configured to move the second plunger within the second cylindrical reservoir in a direction from the second plunger starting point towards the second plunger ending point.

5. The medical dispensing device according to claim 1, wherein the medical dispensing device comprises a first plunger rod that is configured to engage the first plunger, and a second plunger rod that is configured to engage the second plunger, wherein the drive is configured to selectively actuate either the first plunger rod or the second plunger rod.

6. The medical dispensing device according to of claim 1, wherein the drive comprises a hinge and a coupling element, wherein the coupling element is configured to couple the drive alternatively to the first plunger or to the second plunger, and wherein the hinge is configured, to selectively couple by a rotational movement either the coupling element to the first plunger and to decouple the coupling element from the second plunger, or to decouple the coupling element from the first plunger and to couple the coupling element to the second plunger.

7. The medical dispensing device according to claim 1, wherein the drive comprises a sliding mechanism and a coupling element, wherein the coupling element is configured to couple the drive alternatively to the first plunger or the second plunger, and wherein the sliding mechanism is configured, to selectively couple by a sliding movement either the coupling element to the first plunger and to decouple the coupling element from the second plunger, or to decouple the coupling element from the first plunger and to couple the coupling element to the second plunger.

8. The medical dispensing device according to claim 1, wherein the first cylindrical reservoir and the second cylindrical reservoir are oriented antiparallel to each other, wherein the drive comprises a driving element that is for moving the first plunger within in the first cylindrical reservoir in a direction from the first plunger starting point towards the first plunger ending point by a rotational movement in a first direction and that is configured for moving the second plunger within the second cylindrical reservoir in a direction from the second plunger starting point towards the second plunger ending point by a movement in a second direction opposite to the first direction.

9. A medical dispensing system comprising a medical dispensing device according to claim 1, and at least one selected from the group of
a remote control configured to communicate with the medical dispensing device and/or control the medical dispensing device;
an infusion set device configured to couple to the medical dispensing device and wherein the infusion set device is configured to convey liquid medicament received from the medical dispensing device via an invasive infusion cannula into a patient's body;
a body mount configured to attach the medical dispensing device to the skin of a user;
an insertion device configured to insert an invasive infusion cannula at least in part into the patient's body; and
a rechargeable battery.

10. The medical dispensing system according to claim 9, wherein the medical dispensing device is mounted on the infusion set device.
